(19) United States
(12) Patent Application Publication
(10) Pub. No.: US 2003/0069149 A1
(43) Pub. Date: Apr. 10, 2003

(54) Inhibitor for inhibiting carbonaceous powder from heating up/spontaneously igniting and method of inhibiting carbonaceous powder from heating up/spontaneously igniting

(76) Inventors: Yukihiro Adachi, Chiba (JP); Hitoshi Sugawara, Kanagawa (JP); Jin Ogawa, Tokyo (JP)

Correspondence Address: Peter C Schechter, Darby & Darby, 805 third Avenue, New York, NY, 10022-7513, US

(21) Appl. No.: 10/148,770

(22) PCT Filed: Dec. 1, 2000
(86) PCT No.: PCT/JP00/08538

(30) Foreign Application Priority Data
Dec. 2, 1999 (JP) HEI 11-343816
Dec. 2, 1999 (JP) HEI 11-343817

(51) Int. Cl. A61K 7/50

(52) U.S. Cl. 510/155; 510/289; 510/290

(57) ABSTRACT

Provided as a temperature-up·spontaneous ignition inhibitor for carbonaceous powder which can inhibit carbonaceous powder such as coal and carbon blacks from temperature-up and spontaneous ignition in stacking or during storing is (1) a temperature-up·spontaneous ignition inhibitor for carbonaceous powder comprising at least one substance selected from a radical scavenger and an oxygen scavenger or (2) a temperature-up·spontaneous ignition inhibitor for carbonaceous powder comprising an amine base cationic surfactant having a hydrocarbon group having 4 to 22 carbon atoms and a nonionic surfactant. Further, provided is a temperature-up·spontaneous ignition-inhibiting method for carbonaceous powder, characterized by spraying the above temperature-up·spontaneous ignition inhibitor for carbonaceous powder on carbonaceous powder.

### TECHNICAL FIELD

1. Field of the Invention

The present invention relates to a temperature-up(or temperature-elevated)-spontaneous ignition inhibitor for carbonaceous powder which can inhibit carbonaceous powder of coal and carbon blacks from temperature-up and spontaneous ignition in stacking or during storing and a temperature-up-spontaneous ignition-inhibiting method for carbonaceous powder.

2. Background Art

Dust·landslide preventives have so far been used in storing a large amount of coal and carbon blacks in power plants and iron mills in order to prevent generation of dusts and landslide.

A dust preventive is a mixture comprising principally a nonionic surfactant, glycerin and the like, and an aqueous diluent thereof is sprayed on a stack of coal to humidify the sprayed stack of coal, whereby dusts are prevented from being produced.

On the other hand, a landslide preventive comprises an asphalt emulsion, a polymer emulsion and the like, and a solution obtained by diluting it with water is sprayed on a stack of coal to form a cured matter on the surface thereof, whereby landslide in raining is prevented as well as preventing dusts from being produced.

However, the problem of temperature-up of coals and spontaneous ignition following it in addition to dusts and landslide is remained in storing coal.

In particular, when early age coals are stored outside, a stack of coals rises in a temperature in the inside thereof and leads to ignition in a certain case.

The state in the past is that in order to prevent temperature-up, there has been employed a method in which the stacks are destroyed and rebuilt every fixed period or the stacks are pressed in storing coals.

However, the method described above has the problem that a lot of labor is required and the cost is high.

On the other hand, in relation of chemicals with the problem of the spontaneous ignition described above, there has been tried a method in which a large amount of the landslide preventive described above is sprayed to form a solid surface cured matter to thereby prevent spontaneous ignition. However, it has the problems that it is difficult to evenly spray the preventive and cracks produced on the surface a large effect is not obtained because of cracks produced on the surface thereof.

In light of the foregoing problems and existing state of conventional techniques, the present invention intends to solve them, and an object thereof is to provide a temperature-up·spontaneous ignition inhibitor for carbonaceous powder which can inhibit a carbonaceous powder such as coal and carbon blacks from temperature-up and spontaneous ignition in stacking or during storing and a temperature-up·spontaneous ignition-inhibiting method in which the above temperature-up·spontaneous ignition inhibitor is used to inhibit carbonaceous powder from temperature-up and spontaneous ignition.

### DISCLOSURE OF THE INVENTION

Intensive investigations of the problems on the conventional techniques described above repeated by the present inventors have resulted in finding that temperature-up and spontaneous ignition can be inhibited by diluting a specific substance selected from chemical substances with water and/or a suitable organic solvent and spraying it on a carbonaceous powder such as coal and carbon blacks and by diluting a specific amine base cationic surfactant selected from surfactants and a nonionic surfactant with water and/or a suitable organic solvent and spraying it on carbonaceous powder such as coal and carbon blacks, and they have come to complete the present invention.

That is, the present invention comprises the following items (1) to (8).

(1) A temperature-up-spontaneous ignition inhibitor for carbonaceous powder comprising at least one substance selected from a radical scavenger and an oxygen scavenger.

(2) The temperature-up·spontaneous ignition inhibitor for carbonaceous powder as described in the above item (1), wherein the radical scavenger and the oxygen scavenger are selected from hydroquinone, 2,6-di-tertiary-butylhydroquinone, phenol, catechol, p-tertiary-butylcatechol, resorcin, 1-naphthol, pyrogallol, 4-chlororesorcin, aniline, o-aminophenol, p-aminophenol, 2-methyl-5-aminophenol, 4-aminoanisole, 3-hydroxy-4-aminoanisole, p-phenylenediamine, m-phenylenediamine, p-phenetidine, o-tolylenediamine, m-tolylenediamine, 2-chloro-p-phenylenediamine, 4-methoxy-p-phenylenediamine, N,N′-bis(2-hydroxyethyl)-p-phenylenediamine, N,N′-diphenyl-p-phenylenediamine, 2-nitro-o-phenylenediamine, 4-nitro-o-phenylenediamine, tolylenediisocyanate, hydrazine, sodium sulfite, sodium hydrogensulfite, sodium hydrosulfite, sodium thiosulfate, sulfur, 2,6-di-tertiary-butyl-p-cresol, 2-tertiary-butyl-4-methoxyphenol, propyl gallate, isoamyl gallate and ethyl protocatechuate.

(3) The temperature-up-spontaneous ignition inhibitor for carbonaceous powder as described in the above item (1) or (2), further comprising at least one nonionic surfactant.

(4) A temperature-up spontaneous ignition inhibitor for carbonaceous powder comprising an amine base cationic surfactant having a hydrocarbon group having 4 to 22 carbon atoms and a nonionic surfactant.

(5) The temperature-up·spontaneous ignition inhibitor for carbonaceous powder as described in the above items (3) or (4), wherein the nonionic surfactant described above is at least one selected from polyoxyalkylene alkyl ether represented by the following Formula (I) and polyoxyalkylene alkylphenyl ether represented by the following Formula (II):

R₁O—(AO)ₙ—H (I)

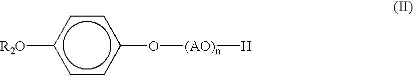

wherein R₁ and R₂ each represent an alkyl group or alkenyl group having 6 to 22 carbon atoms; AO represents oxyethylene and/or oxypropylene; and n is a number of 1 to 100, preferably 3 to 40.

(6) The temperature-up·spontaneous ignition inhibitor for carbonaceous powder as described in the above item (4) or (5), wherein the amine base cationic surfactant described above is at least one selected from a quaternary ammonium salt represented by the following Formula (III) and an amine represented by the following Formula (IV) or a salt thereof:

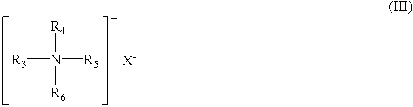

wherein one to three of R₃, R₄, R₅ and R₆ represent an alkyl group or alkenyl group having 4 to 22, preferably 8 to 22 carbon atoms, phenyl or benzyl, and the others represent methyl, ethyl or (CₓH₂ₓO)_{y}OH, provided that x is 2 to 4 and y is 1 to 10; and X⁻ represents a halogen ion such as chlorine, bromine and iodine, or a paired ion such as an ethyl sulfate ion, an acetic acid ion, a hydroxyl ion and a hydrogensulfate ion:

R₇—NH₂ (IV)

wherein R₇ represents an alkyl group or alkenyl group having 4 to 22, preferably 8 to 22 carbon atoms.

(7) A temperature-up·spontaneous ignition-inhibiting method for carbonaceous powder, characterized by spraying the temperature-up·spontaneous ignition inhibitor for carbonaceous powder as described in any of the above items (1) to (6) on carbonaceous powder.

(8) The temperature-up spontaneous ignition-inhibiting method for carbonaceous powder as described in the above item (7), wherein the carbonaceous powder is coal.

The radical scavenger and the oxygen scavenger defined in the present invention mean compounds which react with radicals and oxygen to allow themselves to turn into relatively stable compounds.

### BEST MODE FOR CARRYING OUT THE INVENTION

The respective embodiments shall be explained below in order to describe the present invention in more details.

The temperature-up·spontaneous ignition inhibitor for carbonaceous powder in the first embodiment of the present invention is characterized by comprising at least one substance selected from a radical scavenger and an oxygen scavenger.

The radical scavenger and the oxygen scavenger used in the first embodiment of the present invention react, as described above, with radicals and oxygen to allow themselves to turn into relatively stable compounds. Such compounds include, for example, hydroquinone, 2,6-di-tertiary-butylhydroquinone, phenol, catechol, p-tertiary-butylcatechol, resorcin, 1-naphthol, pyrogallol, 4-chlororesorcin, aniline, o-aminophenol, p-aminophenol, 2-methyl-5-aminophenol, 4-aminoanisole, 3-hydroxy-4-aminoanisole, p-phenylenediamine, m-phenylenediamine, p-phenetidine, o-tolylenediamine, m-tolylenediamine, 2-chloro-p-phenylenediamine, 4-methoxy-p-phenylenediamine, N,N′-bis(2-hydroxyethyl)-p-phenylenediamine, N,N′-diphenyl-p-phenylenediamine, 2-nitro-o-phenylenediamine, 4-nitro-o-phenylenediamine, tolylenediisocyanate, hydrazine, sodium sulfite, sodium hydrogensulfite, sodium hydrosulfite, sodium thiosulfate, sulfur, 2,6-di-tertiary-butyl-p-cresol, 2-tertiary-butyl-4-methoxyphenol, propyl gallate, isoamyl gallate and ethyl protocatechuate.

Among them, preferred from the viewpoints of the cost and the temperature-up·spontaneous ignition-inhibiting effect are hydroquinone, phenol, p-phenylenediamine, m-phenylenediamine, o-tolylenediamine, 2,6-di-tertiary-butyl-p-cresol, 2-tertiary-butyl-4-methoxyphenol, sodium sulfite, sodium thiosulfate and sodium hydrosulfite.

These radical scavengers and oxygen scavengers can be used alone or in combination of two or more kinds thereof.

Further, in the first embodiment of the present invention, nonionic surfactants in addition to these inhibitors can be used alone or in combination of two or more kinds thereof from the viewpoint of further rise in the temperature-up·spontaneous ignition-inhibiting effect.

The nonionic surfactant which can be used in the first embodiment of the present invention shall not specifically be restricted and includes, for example, polyoxyalkylene alkyl ether represented by the following Formula (I) and polyoxyalkylene alkylphenyl ether represented by the following Formula (II):

R₁O—(AO)ₙ—H (I)

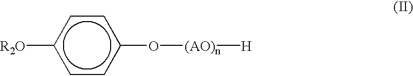

wherein R₁ and R₂ each represent an alkyl group or alkenyl group having 6 to 22 carbon atoms; AO represents oxyethylene and/or oxypropylene; and n is a number of 1 to 100, preferably 3 to 40.

The nonionic surfactant represented by Formula (I) or (II) described above includes, for example, nonylphenol ethylene oxide (7 mole adduct), lauryl alcohol ethylene oxide (7 mole adduct), tridecyl alcohol ethylene oxide (9 mole adduct), pentadecyl alcohol ethylene oxide (9 mole adduct) and coconut alcohol ethylene oxide (9 mole adduct).

A blending amount of this nonionic surfactant is 90 mass % or less (hereinafter mass % is called merely ┌%┘), preferably 70% or less and more preferably 50% or less based on the whole amount of the temperature-up·spontaneous ignition inhibitor.

The above surfactant-containing temperature-up·spontaneous ignition inhibitor solution is improved in permeability into carbonaceous powder such as coal and the like by blending the nonionic surfactant described above, whereby the temperature-up·spontaneous ignition-inhibiting effect can further be elevated.

A reduction in a portion (amount) of the radical scavenger and the oxygen scavenger results in a reduction in the effect to temperature-up-spontaneous ignition inhibition, and therefore it is not preferred to blend the nonionic surfactant in a blending amount exceeding 90%.

Next, the temperature-up spontaneous ignition inhibitor for carbonaceous powder in the second embodiment of the present invention is characterized by comprising an amine base cationic surfactant having a hydrocarbon group having 4 to 22 carbon atoms and a nonionic surfactant.

The amine base cationic surfactant used in the second embodiment of the present invention shall not specifically be restricted as long as it is an amine base cationic surfactant having a hydrocarbon group having 4 to 22 carbon atoms and includes preferably a quaternary ammonium salt represented by the following Formula (III) and an amine represented by the following Formula (IV) or a salt thereof:

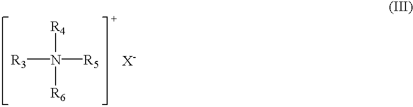

wherein one to three of R₃, R₄, R₅ and R₆ represent an alkyl group or alkenyl group having 4 to 22, preferably 8 to 22 carbon atoms, phenyl or benzyl, and the others represent methyl, ethyl or (CₓH₂ₓO)_{y}OH, provided that x is 2 to 4 and y is 1 to 10; and X⁻ represents a halogen ion such as chlorine, bromine and iodine, or a paired ion such as an ethyl sulfate ion, an acetic acid ion, a hydroxyl ion and a hydrogensulfate ion: wherein R₇ represents an alkyl group or alkenyl group having 4 to 22, preferably 8 to 22 carbon atoms.

The quaternary ammonium salt represented by Formula (III) described above or the amine or the salt thereof

R₇—NH₂ (IV)

represented by Formula (IV) described above includes, for example, cetyltrimethylammonium chloride, (beef tallow)alkyltrimethylammonium chloride, stearyltrimethylammonium bromide, tetrabutylammonium hydrogensulfate, tetrabutylammonium hydroxide, benzyltriethylammonium chloride, dicoconutalkyldimethylammonium chloride, dioleyldimethylammonium chloride, oleyldi(hydroxyethyl)methylammonium chloride, coconutalkylamine acetate, (beef tallow)alkylamine acetate, laurylamine hydrochloride, di(hardened beef tallow)alkyldimethylammonium acetate and trilaurylmethylammonium chloride.

These amine base cationic surfactants can be used alone or in combination of two or more kinds thereof.

The surfactants having a hydrocarbon group having less than 4 carbon atoms has a small adsorptivity to coal. On the other hand, the surfactants having a hydrocarbon group having more than 22 carbon atoms is inferior in affinity to water and/or an organic solvent in diluting and spraying, and therefore homogeneous dilution is difficult. Accordingly, both are not preferred.

The nonionic surfactant which can be used in the second embodiment of the present invention shall not specifically be restricted and includes, for example, polyoxyalkylene alkyl ether represented by Formula (I) described above and polyoxyalkylene alkylphenyl ether represented by Formula (II) described above which were described in details in the first embodiment described above. Various surfactants described in details in the first embodiment described above can be used for the specific nonionic surfactant which can be used in the second embodiment of the present invention, and the explanations thereof shall be omitted.

In the second embodiment of the present invention, a blending ratio of the nonionic surfactant is 10% or more and 90% or less, preferably 70% or less and more preferably 50% or less based on the total amount of the amine base cationic surfactant and the others (the whole amount of the temperature-up·spontaneous ignition inhibitor).

The above temperature-up-spontaneous ignition inhibitor solution is improved in permeability into carbonaceous powder such as coal and the like by blending the nonionic surfactant described above, whereby the temperature-up·spontaneous ignition-inhibiting effect can be elevated.

A reduction in a portion (amount) of the amine base cationic surfactant results in a reduction in the effect to temperature-up·spontaneous ignition inhibition, and therefore it is not preferred to blend the nonionic surfactant in a blending amount exceeding 90%.

The temperature-up·spontaneous ignition inhibitor of the present invention in the first embodiment or the second embodiment thus constituted can be used by diluting with water and/or a suitable organic solvent (diluent solvent), for example, alcohols such as ethanol and the like.

Water and/or the organic solvent used for dilution is used in one to 10,000 times, preferably 10 to 1,000 times and more preferably 20 to 1000 times based on the temperature-up·spontaneous ignition inhibitor.

If the diluent solvent is increased (exceeds 10,000 times), a large amount of the inhibitor has to be sprayed in order to obtain the effect, and therefore it is not preferred. On the other hand, the diluent solvent is small (less than one time), the viscosity grows high, and the handling property is lowered. Accordingly, both are not preferred.

The method of the present invention is characterized by spraying the temperature-up·spontaneous ignition inhibitor in the first embodiment or the second embodiment on carbonaceous powder. To be specific, it can be carried out, as described above, by diluting the temperature-up·spontaneous ignition inhibitors for carbonaceous powder in the respective embodiments with water and/or a suitable organic solvent (diluent solvent) and spraying the solutions on carbonaceous powder.

A spraying amount of the temperature-up·spontaneous ignition inhibitors in the respective embodiments of the present invention described above depends strongly on the properties and the particle size distribution of carbonaceous powder concerned and therefore can not univocally be decided. If the spraying amount is small, the temperature-up·spontaneous ignition inhibition effect can not be expected. On the other hand, if the spraying amount is large, the properties of carbonaceous powder are changed in a certain case.

In the present invention, the carbonaceous powder intended for temperature-up·spontaneous ignition inhibition includes, for example, coals such as anthracite, bituminous coal, sub-bituminous coal and brown coal, petroleum coke, carbon blacks by-produced from chemical plants, carbon blacks obtained by carbonizing organic matters and those used as an energy source such as charcoal. Coals are preferred.

Further, the present invention is used for carbonaceous powder such as coal and the like, and it can be used in or after stacking operation. It is preferred to evenly spray the inhibitor in stacking operation in order to elevate the effect of the present invention. In this case, it can be sprayed by means of a dedicated atomizer or a spraying facility such as a sprinkler and a motor sprayer.

In the temperature-up·spontaneous ignition inhibitor for carbonaceous powder and the temperature-up·spontaneous ignition-inhibiting method therefor in the first embodiment of the present invention, the inhibitor comprising at least one substance selected from the radical scavenger and the oxygen scavenger or at least one nonionic surfactant in addition to them is, as described above, diluted with water and/or a suitable organic solvent and sprayed on carbonaceous powder such as coals and carbon blacks, whereby temperature-up and spontaneous ignition can be inhibited.

The reason why such inhibitor (chemical) has a temperature-up·spontaneous ignition-inhibiting effect is that the radical scavenger or the oxygen scavenger has an action to reduce an amount of radicals produced by the reaction of carbonaceous powder with oxygen and retards oxidation reaction subsequent thereto to inhibit reaction heat from being generated. Conventional chemicals, that is, dust·landslide preventives do not have such action as retarding radicals, but the inhibitor in the first embodiment of the present invention can directly retard oxidation reaction to inhibit temperature-up·spontaneous ignition.

Further, combined use of the nonionic surfactant in the first embodiment described above makes it possible to elevate the permeability into carbonaceous powder and further enhance the effect of the radical scavenger or the oxygen scavenger (these points shall further be explained in examples described later).

In the temperature-up spontaneous ignition inhibitor and the temperature-up·spontaneous ignition-inhibiting method therefor in the second embodiment of the present invention constituted as described above, the temperature-up·spontaneous ignition inhibitor for carbonaceous powder characterized by comprising the amine base cationic surfactant having a hydrocarbon group having 4 to 22 carbon atoms and the nonionic surfactant is diluted with water and/or a suitable organic solvent and sprayed on carbonaceous powder such as coals and carbon blacks, whereby temperature-up and spontaneous ignition can be inhibited.

This inhibitor in the second embodiment has an oxidation reaction-retarding effect as well as a dust-preventing performance with which the amine base cationic surfactant is intrinsically endowed. Further, combined use of the nonionic surfactant makes it possible to elevate the permeability into carbonaceous powder and further enhance the oxidation reaction-retarding effect of the amine base cationic surfactant (these points shall further be explained in examples described later).

### EXAMPLES

Next, the present invention shall more specifically be explained with reference to examples and comparative examples, but the present invention shall not be restricted to the following examples.

### Examples 1 to 9 and Comparative Examples 1 to 3

The temperature-up-spontaneous ignition inhibitors (chemicals) used are shown in the following Table 1, and the nonionic surfactants used are shown in the following Table 2.

The temperature-up-spontaneous ignition inhibitors were prepared according to compositions shown in the following Table 3.

The temperature-up·spontaneous ignition inhibitors obtained in Examples 1 to 9 and Comparative Examples 1 to 3 were subjected to a spontaneous ignition test according to the following test method.

The results obtained are shown in the following Table 3. ┌%┘ in Table 3 is mass %.

Test method of spontaneous ignition:

A ┌spontaneous-ignitable test equipment SIT-2┘ manufactured by Shimadzu Mfg. Co., Ltd. was used for the test equipment.

In respect to the test conditions, precisely weighed was about 900 mg of Asam Asam coal (coal produced in Indonesia) which was crushed into 60 mesh or less and which was sprayed with the chemicals shown in the following Table 3 dissolved in water when soluble in water and in a water and ethanol-mixed (1:1) solvent system when insoluble in water by means of a sprayer, and it was put into a cell in the equipment and left standing at 50° C. for about one hour under nitrogen atmosphere.

Then, dried air was allowed to flow thereinto at a rate of 5 ml/minute to observe a change in the temperature.

In the above procedure, measured was time required for elevating the temperature from 50° C. which was an initial temperature up to 200° C. It is shown that the longer the time required for elevating the temperature is, the more excellent the effect for inhibiting carbonaceous powder from temperature-up spontaneous ignition is.

TABLE 1
Chemicals used

| | | |
|---|---|---|
| Organic | A | Phenol |
| compounds | B | p-Phenylenediamine |
| | C | o-Tolylenediamine |
| | D | Hydroquinone |
| Inorganic | I | Sodium sulfite |
| compounds | J | Sodium thiosulfate |
| | K | Sodium hydrosulfite |

TABLE 2
Nonionic surfactants

| | |
|---|---|
| a | Nonylphenol ethylene oxide (7 mole adduct) |
| b | Lauryl alcohol ethylene oxide (7 mole adduct) |

TABLE 3

| | | | | | Diluting | |
|---|---|---|---|---|---|---|
| | Chemical | | Nonionic surfactant | | magnification | Temperature-up |
| | | Addition amount | | Addition amount | of sprayed | time (min) |
| | Code | (% based on coal) | Code | (% based on coal) | solution | (50° C.→200° C.) |
| Example 1 | A | 1.0 | — | — | 20 | 554 |
| Example 2 | B | 1.0 | — | — | 20 | 566 |
| Example 3 | B | 1.0 | a | 0.4 | 20 | 586 |
| Example 4 | C | 1.0 | — | — | 20 | 552 |
| Example 5 | C | 1.0 | b | 0.4 | 20 | 579 |
| Example 6 | D | 1.0 | b | 0.4 | 20 | 531 |
| Example 7 | I | 1.0 | b | 0.4 | 20 | 560 |
| Example 8 | J | 1.0 | b | 0.4 | 20 | 544 |
| Example 9 | K | 1.0 | a | 0.4 | 20 | 547 |
| Comparative | — | — | — | — | — | 345 |
| Example 1 | | | | | | |
| Comparative | — | — | a | 0.4 | 20 | 403 |
| Example 2 | | | | | | |
| Comparative | — | — | b | 0.4 | 20 | 412 |
| Example 3 | | | | | | |

As apparent from the results shown in Tables 1 to 3, it has been found that the carbonaceous powders sprayed with the temperature-up spontaneous ignition inhibitors can notably be inhibited from temperature-up·spontaneous ignition in Examples 1 to 9 falling in the scope of the present invention as compared with Comparative Examples 1 to 3 falling outside the scope of the present invention.

To specifically observe, the temperature-up time was 345 (minutes) in Comparative Example 1 where no treatment was provided, and the temperature-up times were 403 (minutes) and 412 (minutes) respectively in Comparative Examples 2 and 3 where only the nonionic surfactant solutions were added.

In contrast with this, it has been found that all of the temperature-up times are 500 (minutes) or longer in Examples 1 to 9 falling in the scope of the present invention. Further, respective comparisons of Example 2 with Example 3 and Example 4 with Example 5 have made it clear that the temperature-up spontaneous ignition inhibitors using the surfactants in combination therewith are extended in temperature-up time, that is, excellent in further inhibiting temperature-up·spontaneous ignition.

### Examples 10 to 22 and Comparative Examples 4 to 9

The amine base cationic surfactants used are shown in the following Table 4, and the nonionic surfactants used are shown in the following Table 5.

The temperature-up-spontaneous ignition inhibitors were prepared according to compositions shown in the following Table 6.

The temperature-up·spontaneous ignition inhibitors obtained in Examples 10 to 22 and Comparative Examples 4 to 9 were subjected to the spontaneous ignition test according to the test method used in Example 1 described above. The coal used is Absaloka coal (coal produced in U.S.).

The results obtained are shown in the following Table 6. ┌%┘ in Table 6 is mass %.

TABLE 4
Amine base cationic surfactants used

| | |
|---|---|
| \mathring{1} | Cetyltrimethylammonium chloride |
| \mathring{2} | (Beef tallow)alkyltrimethylammonium chloride |
| \mathring{3} | Stearyltrimethylammonium bromide |
| \mathring{4} | Tetrabutylammonium hydrogensulfate |
| \mathring{5} | Tetrabutylammonium hydroxide |
| \mathring{6} | Benzyltriethylammonium chloride |
| \mathring{7} | Dicoconutalkyldimethylammonium chloride |
| \mathring{8} | Dioleyldimethylammonium chloride |
| \mathring{9} | Oleyldi (hydroxyethyl)methylammonium chloride |
| \mathring{10} | Coconutalkylamine acetate |
| \mathring{11} | (Beef tallow)alkylamine acetate |
| \mathring{12} | Laurylamine hydrochloride |

TABLE 5
Nonionic surfactants

| | |
|---|---|
| a | Nonylphenol ethylene oxide (7 mole adduct) |
| b | Lauryl alcohol ethylene oxide (7 mole adduct) |

TABLE 6

| | | | | | Diluting | |
|---|---|---|---|---|---|---|
| | Cationic surfactant | | Nonionic surfactant | | magnification | Temperature-up |
| | | Addition amount | | Addition amount | of sprayed | time (min) |
| | Code | (% based on coal) | Code | (% based on coal) | solution | (50° C.→200° C.) |
| Example 10 | \mathring{1} | 1.0 | a | 0.4 | 20 | 650 |
| Example 11 | \mathring{2} | 1.0 | b | 0.4 | 20 | 630 |
| Example 12 | \mathring{3} | 1.0 | b | 0.4 | 20 | 684 |
| Example 13 | \mathring{4} | 1.0 | a | 0.4 | 20 | 655 |
| Example 14 | \mathring{5} | 1.0 | a | 0.4 | 20 | 643 |
| Example 15 | \mathring{6} | 1.0 | a | 0.4 | 20 | 660 |
| Example 16 | \mathring{7} | 1.0 | a | 0.4 | 20 | 678 |
| Example 17 | \mathring{8} | 1.0 | b | 0.4 | 20 | 647 |
| Example 18 | \mathring{9} | 1.0 | b | 0.4 | 20 | 643 |
| Example 19 | \mathring{10} | 1.0 | b | 0.4 | 20 | 659 |
| Example 20 | \mathring{11} | 1.0 | a | 0.4 | 20 | 628 |
| Example 21 | \mathring{12} | 1.0 | a | 0.4 | 20 | 631 |
| Comparative | — | — | — | — | — | 430 |
| Example 4 | | | | | | |
| Comparative | — | — | a | 0.4 | 20 | 558 |
| Example 5 | | | | | | |
| Comparative | — | — | b | 0.4 | 20 | 562 |
| Example 6 | | | | | | |
| Comparative | \mathring{1} | 1.0 | — | — | 20 | 607 |
| Example 7 | | | | | | |
| Comparative | \mathring{2} | 1.0 | — | — | 20 | 591 |
| Example 8 | | | | | | |
| Comparative | \mathring{3} | 1.0 | — | — | 20 | 587 |
| Example 9 | | | | | | |

As apparent from the results shown in Tables 4 to 6, it has been found that the carbonaceous powders sprayed with the temperature-up·spontaneous ignition inhibitors can notably be inhibited from temperature-up·spontaneous ignition in Examples 10 to 22 falling in the scope of the present invention as compared with Comparative Examples 4 to 9 falling outside the scope of the present invention.

To specifically observe, the temperature-up time was 430 (minutes) in Comparative Example 4 where no treatment was provided; the temperature-up times were 558 (minutes) and 562 (minutes) respectively in Comparative Examples 5 and 6 where only the nonionic surfactant solutions were added; and the temperature-up times were 607 (minutes), 591 (minutes) and 587 (minutes) respectively in Comparative Examples 7 to 9 where only the amine base cationic surfactant solutions were added.

In contrast with this, it has been found that all of the temperature-up times are 628 (minutes) or longer in Examples 10 to 22 falling in the scope of the present invention.

Accordingly, it has been found that the temperature-up time can be extended only by adding the amine base cationic surfactant having a hydrocarbon group having 4 to 22 carbon atoms and the nonionic surfactant, and therefore they are excellent in inhibiting temperature-up spontaneous ignition of carbonaceous powder.

### INDUSTRIAL APPLICABILITY

As described above, the temperature-up·spontaneous ignition inhibitor for carbonaceous powder and the temperature-up spontaneous ignition-inhibiting method therefor according to the present invention makes it possible to simply and easily inhibit temperature-up·spontaneous ignition in storage by spraying the temperature-up·spontaneous ignition inhibitor on carbonaceous powders such as coal and the like and thus makes it possible to store carbonaceous powders such as coal and the like which are likely to spontaneously ignite over a long period of time and reduce proceedings such as water spraying and restacking in case of temperature-up in stacking or during storing.

1. A temperature-up·spontaneous ignition inhibitor for carbonaceous powder comprising at least one substance selected from a radical scavenger and an oxygen scavenger.

2. The temperature-up spontaneous ignition inhibitor for carbonaceous powder as described in claim 1, wherein the radical scavenger and the oxygen scavenger are selected from hydroquinone, 2,6-di-tertiary-butylhydroquinone, phenol, catechol, p-tertiary-butylcatechol, resorcin, 1-naphthol, pyrogallol, 4-chlororesorcin, aniline, o-aminophenol, p-aminophenol, 2-methyl-5-aminophenol, 4-aminoanisole, 3-hydroxy-4-aminoanisole, p-phenylenediamine, m-phenylenediamine, p-phenetidine, o-tolylenediamine, m-tolylenediamine, 2-chloro-p-phenylenediamine, 4-methoxy-p-phenylenediamine, N,N′-bis(2-hydroxyethyl)-p-phenylenediamine, N,N′-diphenyl-p-phenylenediamine, 2-nitro-o-phenylenediamine, 4-nitro-o-phenylenediamine, tolylenediisocyanate, hydrazine, sodium sulfite, sodium hydrogensulfite, sodium hydrosulfite, sodium thiosulfate, sulfur, 2,6-di-tertiary-butyl-p-cresol, 2-tertiary-butyl-4-methoxyphenol, propyl gallate, isoamyl gallate and ethyl protocatechuate.

3. The temperature-up·spontaneous ignition inhibitor for carbonaceous powder as described in claim 1 or 2, further comprising at least one nonionic surfactant.

4. A temperature-up·spontaneous ignition inhibitor for carbonaceous powder comprising an amine base cationic surfactant having a hydrocarbon group having 4 to 22 carbon atoms and a nonionic surfactant.

5. The temperature-up-spontaneous ignition inhibitor for carbonaceous powder as described in claim 3 or 4, wherein the nonionic surfactant described above is at least one selected from polyoxyalkylene alkyl ether represented by the following Formula (I) and polyoxyalkylene alkylphenyl ether represented by the following Formula (II):
R₁O—(AO)ₙ—H (I) 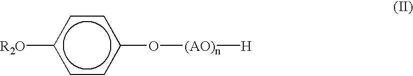
wherein R₁ and R₂ each represent an alkyl group or alkenyl group having 6 to 22 carbon atoms; AO represents oxyethylene and/or oxypropylene; and n is a number of 1 to 100, preferably 3 to 40.

6. The temperature-up·spontaneous ignition inhibitor for carbonaceous powder as described in claim 4 or 5, wherein the amine base cationic surfactant described above is at least one selected from a quaternary ammonium salt represented by the following Formula (III) and an amine represented by the following Formula (IV) or a salt thereof: 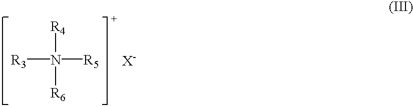
wherein one to three of R₃, R₄, R₅ and R₆ represent an alkyl group or alkenyl group having 4 to 22, preferably 8 to 22 carbon atoms, phenyl or benzyl, and the others represent methyl, ethyl or (CₓH₂ₓO)_{y}OH, provided that x is 2 to 4 and y is 1 to 10; and X⁻ represents a halogen ion such as chlorine, bromine and iodine, or a paired ion such as an ethyl sulfate ion, an acetic acid ion, a hydroxyl ion and a hydrogensulfate ion:
R₇—NH₂ (IV)
wherein R₇ represents an alkyl group or alkenyl group having 4 to 22, preferably 8 to 22 carbon atoms.

7. A temperature-up·spontaneous ignition-inhibiting method for carbonaceous powder, characterized by spraying the temperature-up-spontaneous ignition inhibitor powder as described in any of claims 1 to 6 on carbonaceous powder.

8. The temperature-up spontaneous ignition-inhibiting method for carbonaceous powder as described in claim 7, wherein the carbonaceous powder is coal.
